(19) United States
(12) Patent Application Publication
(10) Pub. No.: US 2022/0117520 A1
(43) Pub. Date: Apr. 21, 2022

(54) BLOOD GLUCOSE SENSOR

(71) Applicant: BIOINTELLISENSE, INC., Redwood City, CA (US)

(72) Inventors: Chi Shin Wang, Los Altos Hills, CA (US); David Jonq Wang, Mountain View, CA (US); Zongde Qiu, Cupertino, CA (US)

(21) Appl. No.: 17/073,943

(22) Filed: Oct. 19, 2020

(51) Int. Cl. A61B 5/145; A61B 5/05

(52) U.S. Cl. CPC A61B 5/14532; A61B 2562/02; A61B 5/0507

(57) ABSTRACT

A method may include generating a frequency-agnostic signal using an adjustable oscillator, and applying the frequency-agnostic signal to a user. The method may also include determining a reflecting value based on the frequency-agnostic signal as reflected by the user. The method may additionally include identifying a peak in the reflecting value by at least repeatedly comparing the determined reflecting value to a previously stored highest reflecting value, and adjusting the adjustable oscillator based on the comparison. The method may also include, after identifying the peak, determining a frequency of the oscillator corresponding to the peak, and outputting the frequency of the oscillator.

### FIELD

The present disclosure generally relates to a blood glucose sensor, and more particularly, some embodiments may relate to a blood glucose sensor that operates from outside of the body.

### BRIEF DESCRIPTION OF RELATED ART

Unless otherwise indicated herein, the materials described herein are not prior art to the claims in the present application and are not admitted to be prior art by inclusion in this section.

Diabetes is a disease that affects almost ten percent of the population in the United States. Monitoring diabetes involves keeping track of blood glucose levels for a user. However, most blood glucose monitors require the user to prick their finger to obtain a drop of blood to check their blood glucose levels. Thus, improvements are needed to blood glucose monitors.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY OF SOME EXAMPLE EMBODIMENTS

This Summary is provided to introduce a selection of concepts in a simplified form that are further described below in the Description. This Summary is not intended to identify key features or essential characteristics of the claimed subject matter, nor is it intended to be used as an aid in determining the scope of the claimed subject matter.

One or more embodiments of the present disclosure may include a method that includes generating a frequency-agnostic signal using an adjustable oscillator, and applying the frequency-agnostic signal to a user. The method may also include determining a reflecting value based on the frequency-agnostic signal as reflected by the user. The method may additionally include identifying a peak in the reflecting value by at least repeatedly comparing the determined reflecting value to a previously stored highest reflecting value, and adjusting the adjustable oscillator based on the comparison. The method may also include, after identifying the peak, determining a frequency of the oscillator corresponding to the peak, and outputting the frequency of the oscillator.

The object and advantages of the embodiments may be realized and achieved at least by one or more of the elements, features, and combinations particularly pointed out in the claims.

It is to be understood that both the foregoing general description and the following detailed description serve as examples and are explanatory and are not restrictive of the invention, as claimed.

### BRIEF DESCRIPTION OF THE DRAWINGS

To further clarify the above and other advantages and features of the present disclosure, a more particular description of the disclosure will be rendered by reference to specific embodiments thereof which are illustrated in the appended drawings. It is appreciated that these drawings depict only typical embodiments of the disclosure and are therefore not to be considered limiting of its scope. The disclosure will be described and explained with additional specificity and detail through the use of the accompanying drawings in which:

FIG. 1 illustrates an example blood glucose monitoring device;

FIG. 2 illustrates another example blood glucose monitoring device;

FIGS. 3A and 3B illustrate a flow chart of an example method of monitoring blood glucose levels;

FIG. 4 illustrates a flow chart of an example method of determining a reflecting value and/or other processing associated with monitoring blood glucose levels; and

FIG. 5 includes a flow chart of an example method of adjusting a step size in monitoring blood glucose levels,

all arranged in accordance with at least one embodiment described herein.

### DETAILED DESCRIPTION OF SOME EXAMPLE EMBODIMENTS

Some embodiments described herein generally relate to monitoring blood glucose levels of a user. Some approaches utilize microwaves or other frequency-based signals to facilitate a non-invasive approach to determining blood glucose levels and/or other chemical concentrations in the body. For example, variations in blood glucose levels may cause a corresponding change in the dielectric constant of the blood. The change in the dielectric constant may be observed by determining a resonant frequency peak of transmitted waves reflected from a user. One approach to providing the microwaves to a user include utilizing a phase-locked loop (PLL) associated with a frequency generating signal such that the PLL may set the frequency of an oscillator and wait for the oscillator to settle on the frequency, then take a reading at that frequency. After a sweep of a set of frequencies is performed, such an approach may identify a peak in the readings across the frequencies. The frequency peak may be applied to a calibrated curve to identify a corresponding blood glucose level. However, such an approach requires the use of PLLs, which may take on the order of tens of milliseconds before the oscillator has settled onto the desired frequency. Additionally, such an approach may utilize a sweep of the potential frequencies corresponding to blood glucose levels in question. For example, if there are one thousand frequencies being checked, it may take tens of seconds before a reading is obtained, with high power usage for that entire span of time. Some embodiments of the present disclosure may overcome one or more of the shortcomings of such PLL-based approaches.

One or more embodiments of the present disclosure may send a frequency-agnostic signal to a user, and sense the reflected signal. The reflected signal may be normalized and/or otherwise processed to determine a reflecting value. The reflecting value may be compared to a previously-stored reflecting value to determine which is higher. Based on the comparison, the input to the oscillator generating the frequency-agnostic signal may be modified. For example, at least some embodiments of the present disclosure may provide a feedback loop to the oscillator to facilitate the oscillator generating a frequency-agnostic signal at a peak of the reflecting value. After reaching the peak (e.g., the comparison keeps shifting back and forth between two values), the frequency of the oscillator may be determined by a counter and an accurate external clock. That frequency may then be used to determine a corresponding blood glucose level. By using such an approach, the feedback loop to adjust the oscillator does not wait like a PLL for the frequency to settle to the desired frequency, permitting much faster determination of the peak in frequency. Additionally, such speed permits less power usage compared to approaches that utilize PLLs.

Reference will now be made to the drawings to describe various aspects of some example embodiments of the disclosure. The drawings are diagrammatic and schematic representations of such example embodiments, and are not limiting of the present disclosure, nor are they necessarily drawn to scale.

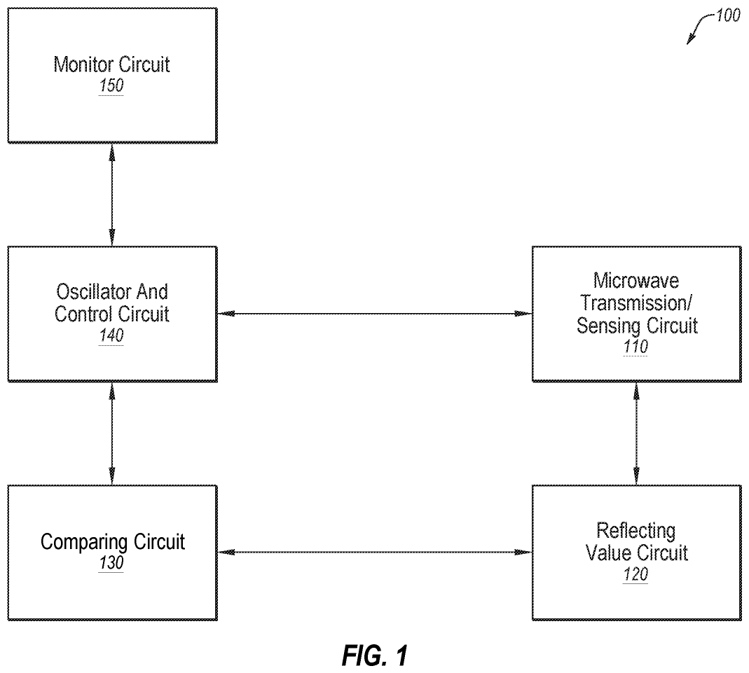

FIG. 1 illustrates an example blood glucose monitoring device 100, in accordance with one or more embodiments of the present disclosure. The blood glucose monitoring device 100 may include a microwave transmission and sensing circuit 110 configured to transmit microwaves into a body of a user and monitor the reflected signals reflected back from the body of the user. The blood glucose monitoring device 100 may include a reflecting value circuit 120 to determine a reflecting value associated with the reflected signals, and a comparing circuit 130 to compare the reflecting value determined by the reflecting value circuit 120 to a previously stored highest reflecting value. The blood glucose monitoring device 100 may include an oscillator and control circuit 140 to generate and control the signal driving the microwave transmissions to the body of the user. The blood glucose monitoring device 100 may include a monitor circuit 150 to determine a frequency of the oscillator of the oscillator and control circuit 140.

In operation, the oscillator and control circuit 140 may begin with a frequency-agnostic signal that is transmitted by the microwave transmission and sensing circuit 110 to the body of the user. The frequency-agnostic signal may be at any frequency and is indeterminate during initial operation of the blood glucose monitoring device 100. The frequency-agnostic signal may include a signal that is not tied to or determined as a particular phase or frequency. For example, in PLL the frequency is determined and although there may be some delay or lag as the PLL settles to the target frequency, the frequency is still set and has a known target. In contrast, a frequency-agnostic signal may be operating at a given frequency, but the blood glucose monitoring device 100 may be agnostic as to the actual value of the frequency and may monitor the relative values of frequency for at least portions of the operation of the blood glucose monitoring device 100.

The microwave transmission and sensing circuit 110 may detect the reflected signal of the initially transmitted signal. For example, the microwave signal (e.g., between about 100 MHz and 100 GHz, between about 100 MHz and 50 GHz, between about 1 GHz and 20 GHz, between about 2 GHz and 10 GHz, between about 4 GHz and 8 GHz, etc.) may be transmitted to and reflected back by the body of the user with variations in the signal based on interactions of the signal with the body, which may be related to various characteristics of the body such as the dielectric constant. The reflected signal may be provided to the reflecting value circuit 120. The reflecting value circuit 120 may use the reflected signal and/or the signal as transmitted to the body to determine a reflecting value. For example, the reflecting value circuit 120 may perform normalization, filtering, processing, calculations, etc. on the reflected signal and/or the signal as transmitted to the body to facilitate determination of the reflecting value. In some embodiments, the reflecting value may include one or more values of a₁, b₁, b₂, S₁₁, S₂₁ or combinations thereof (such as

$$\vert \frac{{b}_{1}}{{a}_{1}} \vert , \vert \frac{{b}_{2}}{{a}_{1}} \vert , {( \frac{{b}_{1}}{{a}_{1}} )}^{2} , {( \frac{{b}_{2}}{{a}_{1}} )}^{2} ,$$

etc.) where a₁ may correspond to an input signal of a first port of a device under test with at least two ports (e.g., a transmitted signal), b₁ may correspond to an output signal of the first port (e.g., a reflected signal), b₂ may correspond to an output signal of a second port of the device under test (e.g., a reflected signal), S₁₁ may correspond to a scattering parameter related to the signals a₁ and b₁ (and may be determined by

$$\frac{{b}_{1}}{{a}_{1}} ) ,$$

and S₂₁ may correspond to a scattering parameter related to the signals a₁ and b₂ (and may be determined by

$$\frac{{b}_{2}}{{a}_{1}} ) .$$

The reflecting value may be provided to the comparing circuit 130.

The comparing circuit 130 may be configured to compare two values, and retain the highest value between the two for future comparisons with an incoming value, and may output a result of the comparison. The output of the comparing circuit 130 may be provided to the oscillator and control circuit 140. The comparing circuit 130 may be configured to compare a currently determined reflecting value with the previously stored highest reflecting value.

The oscillator and control circuit 140 may be configured to receive the output of the comparing circuit 130 and adjust an input to the oscillator accordingly. For example, if the comparing circuit 130 indicates that the previously stored value is higher than the current reflecting value, an input voltage to the oscillator may be increased a set amount and if the current reflecting value is higher than the previously stored value, the input voltage to the oscillator may be decreased a set amount. Additionally or alternatively, if the comparing circuit 130 indicates that the previously stored value is higher than the current reflecting value, the input voltage to the oscillator may be decreased a set amount and if the current reflecting value is higher than the previously stored value, the input voltage to the oscillator may be increased a set amount.

By providing the change to the oscillator, a feedback loop is created that facilitates the oscillator continuing to search to find the frequency corresponding to a peak in the reflecting value using frequency-agnostic frequencies. The peak may be identified based on the comparing circuit 130 repeatedly shifting back and forth between two values. In some embodiments, a threshold number of times shifting back and forth may be detected to signify a peak (such as shifting back and forth between two values at least two times to each value).

After a peak is reached, the monitor circuit 150 may determine the frequency at which the oscillator of the oscillator and control circuit 140 is operating. For example, the monitor circuit 150 may include a counter that uses as its input the output pulses of the oscillator. The counter may utilize an external clock with a high degree of accuracy (such as a quartz crystal clock) to measure the number of pulses in a given unit time such that the counter may output the frequency at which the oscillator is operating at the peak.

In some embodiments, after an initial peak is reached, the oscillator and control circuit 140 may adjust a step-size of the adjustment to the oscillator. For example, the oscillator and control circuit 140 may include a variable resistor located between the output of the comparing circuit 130 and the oscillator such that as the value of the variable resistor is changed, the size of step taken at each comparison may be changed. In these and other embodiments, after the step-size is adjusted, a new peak may be determined. For example, by decreasing the step-size, a higher level of precision and granularity may be achieved as each step moves a smaller distance away from the initially detected peak. The detection of a peak and adjusting the step-size may be repeated a set number of times, until a threshold level of step-size is achieved, or any other metric to arrive at sufficient detail in detecting the final peak. In some embodiments, the frequency of the oscillator may continue to be frequency-agnostic until the final peak is detected, after which, the monitor circuit 150 may determine the frequency of the oscillator corresponding to the final peak. Additionally or alternatively, the frequency of the oscillator may be determined at some or all of the peaks.

In some embodiments, the blood glucose monitoring device 100 may utilize the frequency of the oscillator to determine a blood glucose level of the body of the user. For example, a pre-calibrated curve may associate a set of frequencies with a set of blood glucose levels such that by obtaining the frequency, a blood glucose level may be determined by reference to the pre-calibrated curve.

Modifications, additions, or omissions may be made to the blood glucose monitoring device 100 without departing from the scope of the present disclosure. For example, the components and/or elements of the blood glucose monitoring device 100 may be divided into additional components, combined into fewer components, include additional components, or certain components may be omitted. For example, a microcontroller may be included to facilitate the performance of one or more of the operations and/or tasks described. As another example, one or more of the operations and/or tasks described may be performed via software systems.

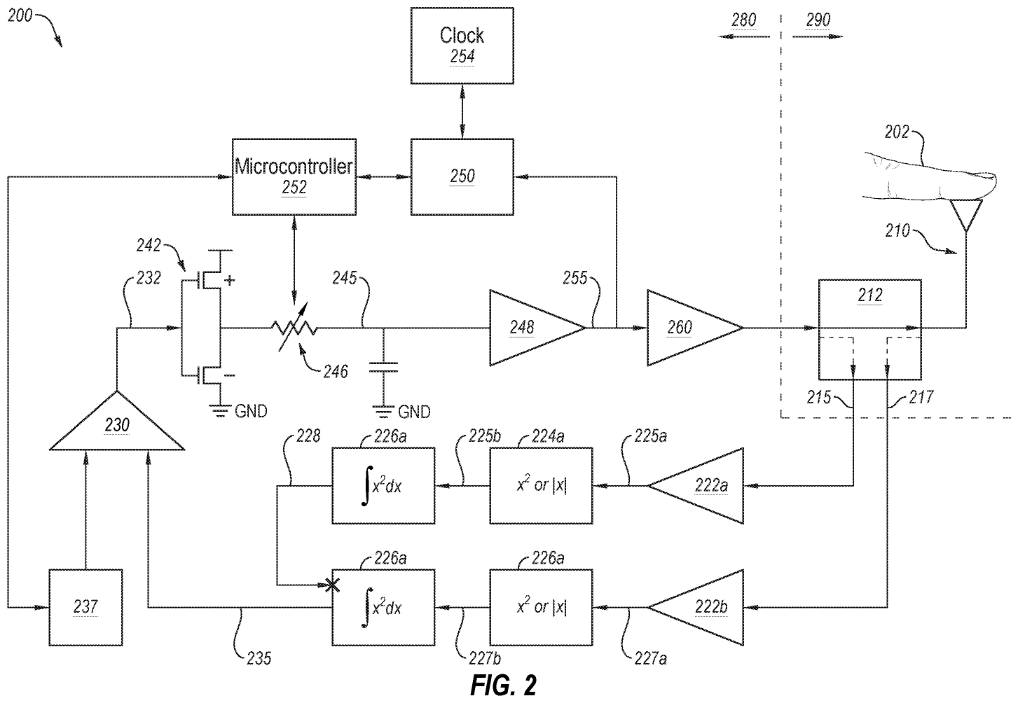

FIG. 2 illustrates another example blood glucose monitoring device 200, in accordance with one or more embodiments of the present disclosure. The blood glucose monitoring device 200 may be similar or comparable to the blood glucose monitoring device 100 of FIG. 1, although FIG. 2 may illustrate one example implementation with additional details. While certain additional details are illustrated in FIG. 2, it will be appreciated that variations and modifications are contemplated as within the scope of the present disclosure. The blood glucose monitoring device 200 may be divided into two general regions, an on-chip region 280 and an off-chip region 290. The blood glucose monitoring device 200 may operate in a similar manner and perform a similar process to that described with reference to the blood glucose monitoring device 100 of FIG. 1. For example, a voltage controlled oscillator 248 may generate a signal that is sent to a body 202 of a user, and the reflection of that signal is used in a feedback loop to adjust the voltage controlled oscillator 248 to identify the peak corresponding to the reflected signal.

The off-chip region 290 may include a coupler 212 that may receive a frequency-agnostic signal 215 that is provided to an antenna 210. The antenna 210 may transmit the signal 215 to the body 202 of the user, and sense the reflecting signal 217. In some embodiments, the coupler 212 may include a bidirectional coupler configured to receive the signal 215 and output it in two places (to the antenna 210 and as an output being sent into the on-chip region 280) and receive the reflected signal 217 from the antenna 210 and output the reflected signal 217 as an output being sent into the on-chip region 280. While the off-chip region 290 illustrates the use of an antenna 210, it will be appreciated that any device may be used to convey the signals to the body 202 and sense the reflected signal, such as a waveguide, etc.

After outputting the initial frequency-agnostic signal 215 and the reflected signal 217, the signals may be normalized. For example the frequency-agnostic signal 215 and/or the reflected signal 217 may be amplified by low noise amplifiers 222a and 222b (respectively) to increase signal while introducing low amounts of noise. The amplified signals 225a and 227a may be passed to mathematical operator components 224a and 224b (respectively). The mathematical operator components 224a and 224b may include any circuitry that may perform a squaring function, an absolute value function, etc. of the amplified signals 225a/227a. In some embodiments, the mathematical operator components 224a and 224b may be implemented as a rectifier circuit, a commutator circuit, etc. The processed signals 225b and 227b may be passed to integral components 226a and 226b, respectively. The integral components 226a and 226b may be configured to integrate values over time until signaled to stop integrating interrupt the integration. The integral component 226a may be configured to integrate the processed signal 225b as it is received until a target value is reached. After reaching the target value, the integral component 226a may output a stop signal 228 to signal the integral component 226b to stop integrating. After being signaled to stop integrating, the integral component 226b may output the integrated signal as a reflecting value 235.

By integrating via the integrating component 226a until a target value is reached, the blood glucose monitoring device 200 may be able to determine the reflecting value with analog circuits and/or components and avoiding an analog digital conversion to perform division. For example, if the reflecting value 235 relates to S₁₁, the denominator in determining Sn may be avoided because the denominator (relating to the input signal, or the frequency-agnostic signal 215) is always the same value and so may be discarded or ignored. In some embodiments, if the reflecting value 235 relates to other values such as b₁ or b₂, the circuitry related to the frequency-agnostic signal 215 (such as the low noise amplifier 222a, the mathematical operator component 224, and/or the integral component 226a) may be omitted. The integral component 226b may be modified to integrate a set number of times or for a set duration of time before outputting the reflecting value 235.

The comparator 230 may be configured to compare the current reflecting value 235 with a previously stored largest reflecting value 237. The comparator 230 may store whichever is larger as the new stored largest reflecting value 237. The output 232 of the comparator 230 may be provided to a component 242. If the output 232 of the comparator is 1 (e.g., the reflecting value 235 is larger than the previously highest reflecting value 237), the component 242 may increase the voltage on the line going to a voltage controlled oscillator 248 generating the frequency-agnostic signal 215. If the output 232 of the comparator is 0 (e.g., the reflecting value 235 is smaller than the previously highest reflecting value 237), the component 242 may decrease the voltage on the line going to a voltage controlled oscillator 248 generating the frequency-agnostic signal 215. Additionally or alternatively, if the output 232 is 1, the component 242 may decrease the voltage and if the output 232 is 0, the component 242 may increase the voltage.

The output of the component 242 may pass through a variable resistor 246 to provide the input 245 to the voltage controlled oscillator 248. For example, the output of the component 242 may increase or decrease the input voltage 245 of the voltage controlled oscillator 248, thereby changing the frequency at which the voltage controlled oscillator 248 is generating an output signal 255 upwards or downwards. The output of the voltage controlled oscillator 255 may be passed to a buffer amplifier 260 to amplify the signal to be output from the on-chip region 280 to the off-chip region 290 and the antenna 210 via the coupler 212 to transmit the signal to the body 202 of the user.

In operation, use of the comparator 230 and the component 242 to vary the voltage controlled oscillator 248 may provide a feedback loop via which the frequency of the voltage controlled oscillator 248 is modified until a peak is identified. After identifying the peak, the output 255 of the voltage controlled oscillator 248 may be provided to a resettable counter 250 that may count the number of pulses of the voltage controlled oscillator 248 for a given unit time as set by an external clock 254.

In some embodiments, a microcontroller 252 may facilitate the operation of various components of the blood glucose monitoring device 200. For example, the microcontroller 252 may be configured to adjust the variable resistor 246. Adjusting the variable resistor 246 may change the step size of the voltage controlled oscillator for each output of the comparator 230. For example, the microcontroller 252 may determine that the comparator 230 is shifting back and forth between two values, and may adjust the variable resistor 246 such that the step size of the voltage changed due to the component 242 is smaller. In these and other embodiments, the microcontroller may trigger the resettable counter 250 to determine the frequency after a final peak is determined subsequent to the variable resistor 246 changing the step size a number of times, to a threshold size, or some other metric and intermediate peaks are determined. In some embodiments, the comparator 230 may be configured to adjust the variable resistor 246 after shifting back and forth between values. In some embodiments, the variable resistor 246 may include a resistor network where various components are activated or deactivated to change the resistance of the variable resistor 246.

In some embodiments, the microcontroller 252 may be configured to store the highest reflecting value and recall or otherwise provide the highest reflecting value to the comparator 230. Additionally or alternatively, the microcontroller 252 may store a pre-calibrated curve such that the microcontroller 252 may obtain the frequency of the peak as output by the resettable counter 250 and output a blood glucose level for the user. For example, the blood glucose level may be output as a blood glucose concentration in mg/dL.

In some embodiments, the on-chip region 280 may be implemented as a series of analog components and/or circuits. In some embodiments, the on-chip region 280 may be implemented as a single integrated circuit and/or chip. For example, the on-chip region 280 may be implemented by a 100 nm radio-frequency complementary metal-oxide-semiconductor (RF CMOS) chip. By using analog components, the blood glucose monitoring device 200 may react quickly. For example, the voltage controlled oscillator 248 may vary its frequency on the order of nanoseconds (such as within 10 ns, within 20 ns, within 50 ns, within 100 ns, etc.), allowing the voltage controlled oscillator 248 to identify the peak very quickly, including changing the step size and re-identifying the peak. By using such an approach, the blood glucose monitoring device 200 may determine a blood glucose level in less than one second, less than half of a second, less than 100 milliseconds, or less than 10 milliseconds. Additionally or alternatively, because of the rapidity with which the blood glucose monitoring device 200 may be able to determine the blood glucose level, the blood glucose monitoring device 200 may utilize less power than PLL devices. Such a decrease in power and/or the use of analog components may facilitate the use of a small device that may be worn for long periods of time without replacing and/or recharging the battery, such as multiple days, multiple weeks, multiple months, etc.

Modifications, additions, or omissions may be made to the blood glucose monitoring device 200 without departing from the scope of the present disclosure. For example, the components and/or elements of the blood glucose monitoring device 200 may be divided into additional components, combined into fewer components, include additional components, or certain components may be omitted. For example, one or more of the operations and/or tasks described may be performed via software systems rather than physical components (e.g., one or more of the signals may be converted to a digital signal such that the operations and/or tasks may be performed via software).

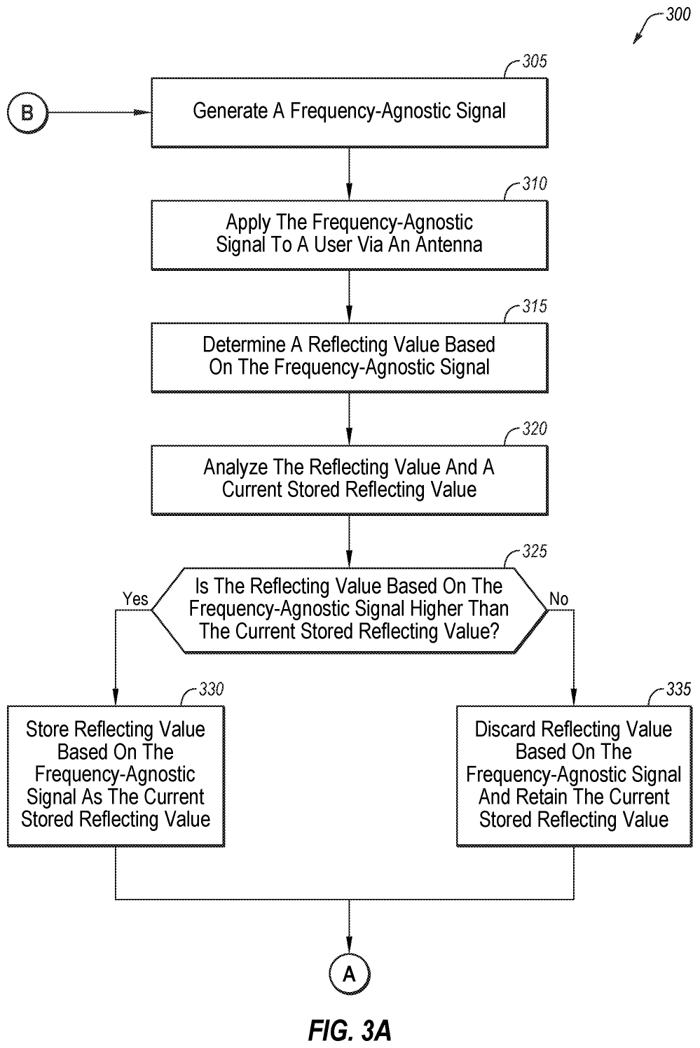

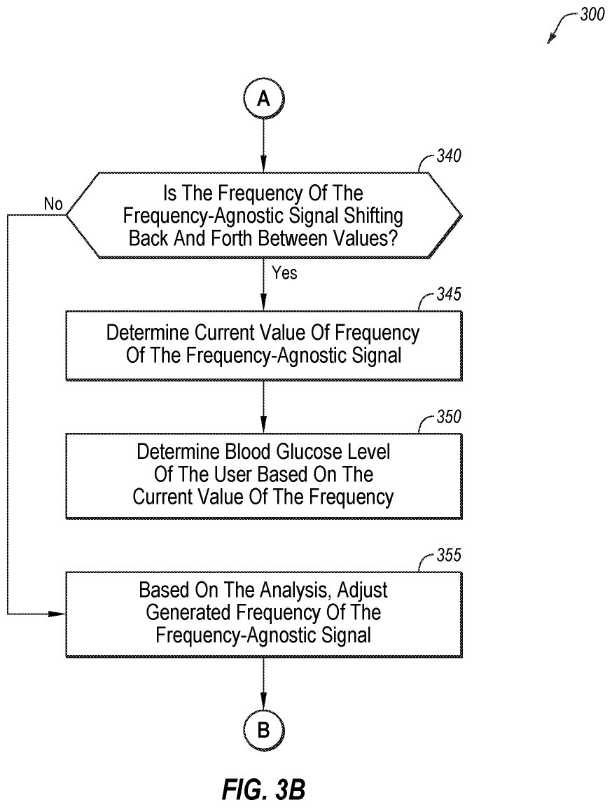

FIGS. 3A and 3B illustrate a flow chart of an example method 300 of monitoring blood glucose levels, in accordance with one or more embodiments of the present disclosure. The method 300 may be performed by any suitable system, apparatus, or device. For example, the blood glucose monitoring device 100 and/or the blood glucose monitoring device 200 of FIGS. 1 and 2, respectively, may perform one or more of the operations associated with the method 300. Although illustrated with discrete blocks, the steps and operations associated with one or more of the blocks of the method 300 may be divided into additional blocks, combined into fewer blocks, or eliminated, depending on the particular implementation.

At block 305, a frequency-agnostic signal may be generated. For example, a voltage controlled oscillator may generate a signal without constraint or control of frequency or phase locking or tracking. In some embodiments, the frequency-agnostic signal may be within a known range (e.g., between 1 GHz and 10 GHz) that may be decided by the physical properties of the voltage controlled oscillator, but may be unknown within that range. In some embodiments, the voltage controlled oscillator may include a quadrature voltage controlled oscillator that may be configured to generate two sinusoidal waves with ninety degrees difference in phase. In these and other embodiments, the waveforms and/or other signals may be represented by real values or by complex values (e.g., a combination of real and imaginary components).

At block 310, the frequency-agnostic signal may be applied to a user via an antenna. For example, the output signal of the voltage controlled oscillator may be amplified and/or buffered and may be provided to the antenna for transmission to the body of the user. In some embodiments, the antenna may be part of a small wearable device that places the antenna against the body of the user. In some embodiments, some other device such as a waveguide may be used to apply the signal to the body of the user instead of or in addition to the antenna.

At block 315, a reflecting value may be determined based on the frequency-agnostic signal. For example, the reflecting value may be any values related to the reflection of the frequency-agnostic signal from the user, such as one or more values of a₁, b₁, b₂, S₁₁, S₂₁ or combinations thereof (such as

$$\vert \frac{{b}_{1}}{{a}_{1}} \vert , \vert \frac{{b}_{2}}{{a}_{1}} \vert , {( \frac{{b}_{1}}{{a}_{1}} )}^{2} , {( \frac{{b}_{2}}{{a}_{1}} )}^{2} ,$$

etc.). In some embodiments, determining the reflecting value may include one or more mathematical operations, such as squaring, absolute values, etc. In some embodiments, the reflection of the frequency-agnostic signal from the user may include both real and imaginary components. For example, the output of the voltage controlled oscillator may include a value such as A cos ωt, where A is the amplitude, ω is related to the period (T) by T=2π/ω, and t is the time. When considering complex values and a quadrature voltage controlled oscillator, the output of the quadrature voltage controlled oscillator may include Ae^{iωt}, or A_{R} cos at +iA_{I} sin ωt, where A_{R} may correspond to the amplitude of the real number portion of the signal, and A_{I} may correspond to the amplitude of the imaginary number portion of the signal. When taking the square of the amplitude of the waveform in real signals (e.g., |A²|), the corresponding complex form includes A_{R}²+A_{I}², leading to A corresponding to \sqrt{A_{R}²+A_{I}²}. Additionally, the absolute value of A may correspond to −|Ae^{i(ωt+φ)}|, where φ may correspond to the phase of the signal.

In some embodiments, determining the reflecting value may include integrating the received signals over time for the reflected signal and/or the transmitted signal. In these and other embodiments, the reflected signal and the transmitted signal may begin integrating at the same time, and may cease based on the integration of the transmitted signal reaching a target value, triggering a stop signal to be sent to stop the integration of the reflected signal.

At block 320, the reflecting value determined at the block 315 may be analyzed in conjunction with a current stored reflecting value. For example, a comparison may be made of the reflecting value determined at the block 315 and the current stored reflecting value to facilitate a determination of which is larger.

At block 325, a determination may be made whether the reflecting value based on the frequency-agnostic signal (e.g., the reflecting value determined at the block 315) is higher than the current stored reflecting value. If the reflecting value based on the frequency-agnostic signal is higher, the method 300 may proceed to the block 330. If the reflecting value based on the frequency-agnostic signal is not higher, the method 300 may proceed to the block 335.

At block 330, the reflecting value based on the frequency-agnostic signal (e.g., the reflecting value determined at the block 315) may be stored as the current stored reflecting value. Such an operation may update the current stored reflecting value to reflect the higher of the reflecting values in the analysis of the block 320. After the block 330, the method 300 may proceed to the block 340.

At block 335, the reflecting value based on the frequency-agnostic signal (e.g., the reflecting value determined at the block 315) may be discarded and the current stored reflecting value may be retained. Such an operation may maintain the current stored reflecting value as the higher of the reflecting values in the analysis of the block 320. After the block 335, the method 300 may proceed to the block 340.

At block 340, a determination may be made whether the frequency of the frequency-agnostic signal is shifting back and forth between values. For example, a determination may be made whether the oscillator generating the signal at the block 305 is repeatedly shifting back and forth between frequencies for the frequency-agnostic signal. The shifting of back and forth between values may signify that the feedback loop to adjust the oscillator may have identified a peak. If it is determined that the frequency of the frequency-agnostic signal is shifting back and forth between values, the method 300 may proceed to the block 345. If it is determined that the frequency of the frequency-agnostic signal is not shifting back and forth between values, the method 300 may proceed to the block 355.

At block 345, the current value of the frequency of the frequency-agnostic signal may be determined. For example, a counter, using a clock, may count the number of pulses of the oscillator for a given unit time when the oscillator is at the peak. Doing so may identify the frequency corresponding to the peak without knowing the frequency of the oscillator beforehand when detecting the presence of the peak. In these and other embodiments, the peak may correspond to a resonant frequency peak.

At block 350, a blood glucose level of the user may be determined based on the current value of the frequency as determined at the block 345. For example, the determined frequency may be compared to a pre-calibrated curve, look-up table, etc. via which the blood glucose level of the user may be correlated with a given resonant frequency peak as determined at the block 345. The blood glucose level may be output or otherwise communicated to the user or a clinician associated with the user.

At block 355, based on the analysis of the block 320 and/or the block 340, the generated frequency of the frequency-agnostic signal may be adjusted. For example, the input voltage to a voltage controlled oscillator may be increased or decreased. In these and other embodiments, the adjustment may be based on the output of the analysis performed at the block 320. After the block 355, the method 300 may return to the block 305. By returning to the block 305, a feedback loop may be utilized to adjust the frequency of the frequency-agnostic signal until a peak is reached, as detected by the block 340 when shifting back and forth between values. In these and other embodiments, such a feedback loop may facilitate the identification of the peak and the corresponding frequency and blood glucose level in a rapid manner, such as in less than one second. In contrast, a PLL-based approach may take around 10 ms to set the accurate frequency, performed for 1000 different frequencies, which may require ten seconds to obtain one blood glucose reading, and may require power consumption for the entire ten seconds. Some embodiments of the present disclosure may obtain the same information in less than one second and use power for the limited time (e.g., less than one second) to obtain the blood glucose level.

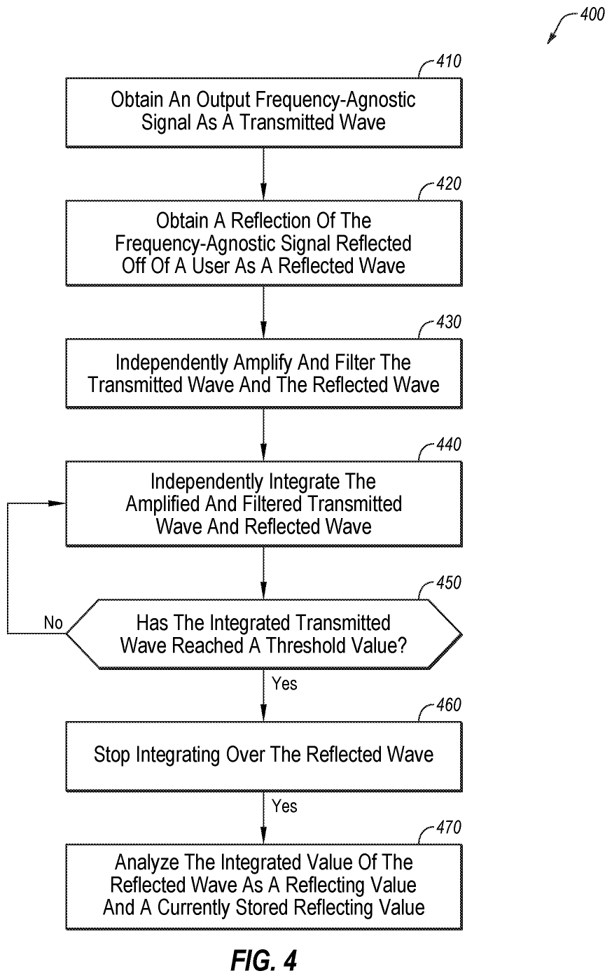

FIG. 4 illustrates a flow chart of an example method 400 of determining a reflecting value and/or other processing associated with monitoring blood glucose levels, in accordance with one or more embodiments of the present disclosure. The method 400 may be performed by any suitable system, apparatus, or device. For example, the blood glucose monitoring device 100 and/or the blood glucose monitoring device 200 of FIGS. 1 and 2, respectively, may perform one or more of the operations associated with the method 400. Although illustrated with discrete blocks, the steps and operations associated with one or more of the blocks of the method 400 may be divided into additional blocks, combined into fewer blocks, or eliminated, depending on the particular implementation. In some embodiments, the method 400 may operate in conjunction with and/or in place of certain operations of the method 300 of FIGS. 3A/3B. For example, the blocks 410-460 may be another example of the block 315, and the operation 470 may be another example of the block 320 of FIG. 3A.

At block 410, an output frequency-agnostic signal may be obtained as a transmitted wave. For example, the transmitted wave may be obtained via a bi-directional coupler in communication with the output of a voltage controlled oscillator.

At block 420, a reflection of the frequency-agnostic signal reflected from a user may be obtained as a reflected wave. For example, the frequency-agnostic signal may be applied to a body of a user via an antenna, waveguide, etc. and the reflection of the transmitted signal may be detected and/or measured.

At block 430, the transmitted wave and the reflected wave may be independently amplified and/or filtered. For example, a low-noise amplifier may amplify either or both of the signals. As another example, either or both of the transmitted wave and the reflected wave may be squared and/or the absolute value thereof may be obtained.

At block 440, the amplified and filtered transmitted wave and/or reflected wave may be independently integrated. For example, the output(s) of the block 430 may be independently integrated over time.

At block 450, a determination may be made whether the integrated transmitted wave has reached a threshold value. For example, a target value may be set for each pass through a feedback loop such that integrating may progress to the same point (reaching the threshold value) each time through the feedback loop. If the threshold value has been reached, the method 400 may proceed to the block 460. If the threshold value has not been reached, the method 400 may return to the block 440 to continue to integrate values over time until the threshold value is reached.

At block 460, the integrating over time of the reflected wave may be stopped. For example, a stop signal may be sent to the component or circuitry performing the integrating of the reflected wave based on the threshold value being reached at the block 450. The result of the integrating over time of the reflected wave may be a reflecting value. In some embodiments, the blocks 410-460 may be one example of the block 315 of FIG. 3A.

At block 470, the integrated value of the reflected wave as a reflecting value may be analyzed in conjunction with a currently stored reflecting value. For example, the reflecting value may be compared to the currently stored reflecting value to determine which is higher. In some embodiments, the block 470 may be similar or comparable to the block 325 of FIG. 3A.

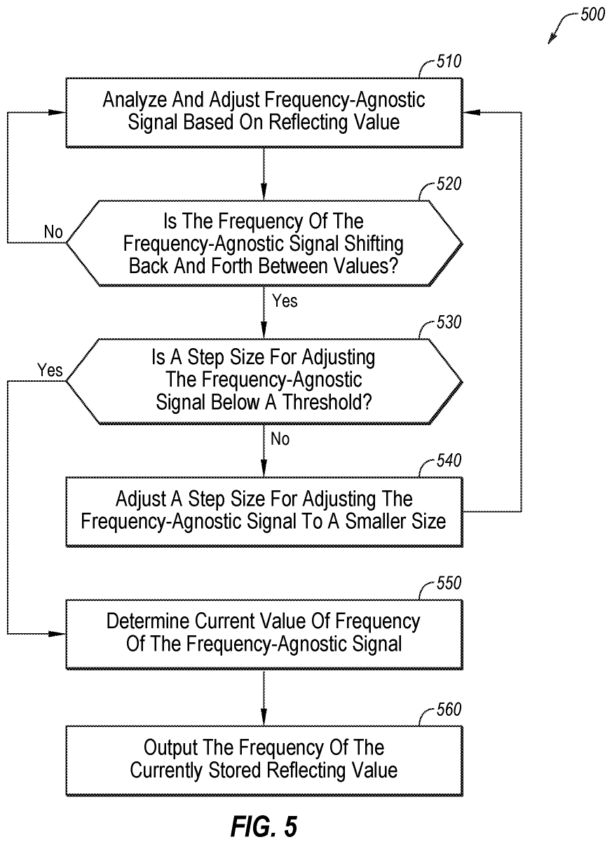

FIG. 5 includes a flow chart of an example method 500 of adjusting a step size in monitoring blood glucose levels, in accordance with one or more embodiments of the present disclosure. The method 500 may be performed by any suitable system, apparatus, or device. For example, the blood glucose monitoring device 100 and/or the blood glucose monitoring device 200 of FIGS. 1 and 2, respectively, may perform one or more of the operations associated with the method 500. Although illustrated with discrete blocks, the steps and operations associated with one or more of the blocks of the method 500 may be divided into additional blocks, combined into fewer blocks, or eliminated, depending on the particular implementation. In some embodiments, the method 500 may work in conjunction with and/or replace certain elements of FIGS. 3A/3B.

At block 510, a frequency-agnostic signal may be analyzed and/or adjusted based on reflecting values. For example, a feedback loop may be used to adjust a control signal for an oscillator to adjust the oscillator based on a comparison of previously stored reflecting values with the currently determined reflecting values. In some embodiments, the block 510 may be similar or comparable to the blocks 305-335 (which may include some or all of the elements of FIG. 4).

At block 520, a determination may be made whether the frequency of the frequency-agnostic signal is shifting back and forth between values. If the frequency is shifting back and forth between values, the method 500 may proceed to the block 530. If the frequency is not shifting back and forth between values, the method 500 may return to the block 510. In some embodiments, the block 520 may be similar or comparable to the block 340 of FIG. 3B.

At block 530, a determination may be made whether a step size for adjusting the frequency-agnostic signal is below a threshold. For example, a determination may be made whether, upon each pass through the feedback loop, the step size is a large step or is a small step (e.g., is below the threshold). In some embodiments, when the step size is based on a variable resistor between the output of circuitry performing the comparison and a voltage controlled oscillator, a value of resistance of the variable resistor may be used as indicative of the step size. If the step size is not below the threshold, the method 500 may proceed to the block 540. If the step size is below the threshold, the method 500 may proceed to the block 550.

At block 540, a step size for adjusting the frequency-agnostic signal may be adjusted to a smaller size. Continuing the example of the variable resistor, the resistance may be increased and/or decreased (depending on the arrangement of the circuit) such that the change in voltage as input to the voltage controlled oscillator in each pass through the feedback loop may be decreased. In this way, after an initial peak is found via the feedback loop, a search may be performed around that peak with a smaller step size to identify a more precise peak. After the block 540, the method 500 may return to the block 510 to search again for a peak based on the frequency-agnostic signal.

At block 550, based on the step-size for adjusting the frequency being below the threshold, the current value of the frequency of the frequency-agnostic signal may be determined. The block 550 may be similar or comparable to the block 345. For example, a counter and/or clock may be used to determine a number of pulses of the oscillator per given unit time. In some embodiments, the frequency of the oscillator may be the frequency corresponding to the currently stored reflecting value, as that value may represent the highest peak reached by the feedback loop.

At block 560, the frequency of the currently stored reflecting value may be output. For example, the frequency determined at the block 550 may be output. Additionally or alternatively, a blood glucose level corresponding to the frequency may be determined and output instead of and/or in addition to the frequency.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

While reference is made in the present disclosure to a peak, and highest/lowest values, it will be appreciated that such values may be dependent on the orientation and/or design of the device implementing one or more embodiments of the present disclosure. For example, a peak may include a lowest value rather than a highest value numerically. As another example, such values may be relative, rather than absolute. For example, the peak may be a peak as related to other data points observed, and not an absolute highest peak.

The present disclosure is not to be limited in terms of the particular embodiments described herein, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that the present disclosure is not limited to particular methods, reagents, compounds, compositions, or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” The terms “about” or “approximately” may include within 10% of a value, for example, “about 5” may include 4.5 to 5.5.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed is:

1. A method, comprising:
generating a frequency-agnostic signal using an adjustable oscillator;
applying the frequency-agnostic signal to a user;
determining a reflecting value based on the frequency-agnostic signal as reflected by the user;
identifying a peak in the reflecting value, including:
repeatedly comparing the determined reflecting value to a previously stored highest reflecting value; and
adjusting the adjustable oscillator based on the comparison;
after identifying the peak, determining a frequency of the oscillator corresponding to the peak; and
outputting the frequency of the oscillator.

2. The method of claim 1, wherein the determining the frequency of the oscillator is performed by a resettable counter measuring a number of pulses of the oscillator in a given unit time as defined by a clock.

3. The method of claim 1, further comprising determining a blood glucose level of the user based on the output frequency of the oscillator and a pre-calibrated curve.

4. The method of claim 1, wherein identifying the peak further comprises, based on the determined reflecting value being higher than the previously stored highest reflecting value, storing the determined reflecting value as the previously stored highest reflecting value.

5. The method of claim 1, wherein adjusting the adjustable oscillator is performed in less than one millisecond.

6. The method of claim 1, wherein identifying the peak further comprises:
determining whether the oscillator is shifting back and forth between values when adjusting the adjustable oscillator; and
based on a step size for adjusting the oscillator being above a threshold and in response to determining the oscillator is shifting back and forth between values, modifying an input to the adjustable oscillator such that the step size is smaller than before modifying the input.

7. The method of claim 6, wherein modifying the input comprises increasing resistance of a variable resistor.

8. The method of claim 1,
wherein the frequency-agnostic signal is a first signal and a reflection of the frequency-agnostic signal reflected from the user is a second signal, and
wherein determining the reflecting value comprises:
amplifying the first signal and the second signal;
performing one of a squaring function or an absolute value function on the first signal and the second signal;
integrating the first signal over time until a target value is reached; and
integrating the second signal over time until the first signal reaches the target value; and
wherein comparing the determined reflecting value includes comparing the integrated second signal to the previously stored highest reflecting value.

9. The method of claim 8, wherein the first signal and the second signal include a real number portion and an imaginary number portion.

10. A device, comprising:
an oscillator configured to generate a frequency-agnostic signal at a given frequency;
a microwave generating and sensing circuit configured to transmit the frequency-agnostic signal to a user;
a reflecting value circuit configured to determine a reflecting value of the frequency-agnostic signal based on a reflection of the frequency-agnostic signal from the user;
a comparing circuit configured to compare the reflecting value of the frequency-agnostic signal with a currently stored reflecting value;
a control circuit configured to change the given frequency of the frequency-agnostic signal generated by the oscillator in response to the comparison performed by the comparing circuit; and
a monitor circuit configured to, based on the control circuit shifting the frequency of the oscillator back and forth between values, determine a frequency of the oscillator and output the frequency of the oscillator.

11. The device of claim 10, wherein at least the oscillator, reflecting value circuit, comparing circuit, and control circuit are analog circuits on a single chip.

12. The device of claim 10, wherein the oscillator includes a voltage-controlled oscillator and a frequency of the voltage-controlled oscillator is undetermined until after the control circuit is shifting the frequency of the oscillator back and forth between values.

13. The device of claim 10, further comprising a processing device configured to extrapolate a blood glucose concentration of the user based on the output of the frequency of the oscillator.

14. The device of claim 10, wherein the reflecting value circuit is further configured to normalize the reflection of the frequency-agnostic signal from the user.

15. The device of claim 14, further comprising:
circuitry to perform either a squaring of the frequency-agnostic signal as a first signal and the reflection of the frequency-agnostic signal from the user as a second signal, or an absolute value of the first signal and the second signal.

16. The device of claim 15, further comprising:
first circuitry to integrate the first signal over time until a target value is reached; and
second circuitry to integrate the second signal over time until a stop signal is received based on the target value being reached by the first circuitry.

17. The device of claim 10, wherein the control circuit includes circuitry to adjust a voltage acting as an input to the oscillator based on a result of the comparing circuit.

18. The device of claim 17, wherein the control circuit includes a variable resistor adjustable by the control circuit, the control circuit further configured to adjust the variable resistor when the control circuit is shifting the oscillator back and forth between two values such that input to the oscillator causes a smaller change in the oscillator than before the adjustment to the variable resistor.

19. The device of claim 10, wherein the monitor circuit includes:
a resettable counter; and
a clock,
wherein the monitor circuit is configured to use the resettable counter to identify a number of oscillations per unit time as determined by the clock to determine the frequency of the oscillator.

20. A device to facilitate measurement of concentrations of chemicals in a human body, comprising:
a microwave device configured to send microwaves of a given frequency to a human body and receive reflections of the microwaves sent to the human body;
a voltage controlled oscillator configured to generate the given frequency at which the microwave device sends the microwaves to the human body based on an input voltage, wherein the voltage controlled oscillator is not controlled by frequency or phase tracking based off of a tracking clock or frequency or phase locking based off of a locking clock;
a frequency tracking feedback loop configured to search for a maximum or minimum point of a reflecting value by adjusting the input voltage of the voltage controlled oscillator, the reflecting value including at least one of an absolute value of a ratio of an reflected wave from the human body a transmitted wave to the human body, the absolute value of the reflected wave from the human body;
a counting circuit configured to determine the given frequency of the voltage controlled oscillator after the maximum or minimum point is achieved, the counting circuit comprising:
a resettable counter using an output of the voltage controlled oscillator as an input pulse; and
an external clock to determine a counting interval of the resettable counter.
